(19) United States
(12) Patent Application Publication
(10) Pub. No.: US 2005/0245938 A1
(43) Pub. Date: Nov. 3, 2005

(54) Method and apparatus for minimally invasive repair of intervertebral discs and articular joints

(76) Inventor: Jeffrey P. Kochan, Penn Valley, PA (US)

Correspondence Address: DUANE MORRIS LLP, PO BOX 5203, PRINCETON, NJ, 08543-5203, US

(21) Appl. No.: 11/067,524

(22) Filed: Feb. 25, 2005

Related U.S. Application Data
(63) Continuation-in-part of application No. 10/834,732, filed on Apr. 28, 2004.

(51) Int. Cl. A61B 17/58

(52) U.S. Cl. 606/92; 604/99.03; 623/17.16

(57) ABSTRACT

A device for repair of intervertebral discs and cartilages in articular joints includes a catheter for inserting through a cannula, the catheter having a distal end and a proximal end and a lumen extending longitudinally therethrough. An expandable balloon may optionally be detachably attached to the catheter near the distal end. The proximal end of the catheter is coupled to an injector that holds a supply of a thermoplastic elastomer material at a predetermined elevated temperature sufficiently high to maintain the thermoplastic elastomer at a liquid state. The device allows a thermoplastic elastomer material to be injected into the intervertegral disc space or the articular joint space as a replacement prosthetic for the disc's nucleus pulposus or the joint's cartilage. This procedure is carried out percutaneously through the cannula.

### CROSS-REFERENCE TO RELATED APPLICATIONS

This application is a continuation-in-part of U.S. patent application Ser. No. 10/834,732, filed on Apr. 28, 2004.

### FIELD OF THE INVENTION

The present invention is in the field of medical devices and methods, and particularly in the field of devices and methods for minimally invasive repair of intervertebral discs and cartilages in articular joints.

### BACKGROUND

The successful repair of diseased or degenerative vertebral discs is extremely important to reducing pain and restoring mobility of the patient. An example of such procedures is the replacement of deteriorated spinal discs by artificial spinal disc implants. In such procedures, an open surgical procedure has been employed to remove the existing deteriorated spinal disc. An artificial disc is then inserted in the space formed by the removal of the deteriorated spinal disc. The artificial disc should occupy a volume equal to that of a healthy disc.

In other known methods, a thermoplastic material maybe injected into the intervertebral disk to replace nucleus pulposus. Such procedures are described, for example, in U.S. Pat. No. 6,436,143 (Ross). The Ross patent describes a procedure where gutta percha based thermoplastic material is injected within the annulus fibrosus to replace the removed nucleus pulposus. In the Ross patent, the thermoplastic material is injected using a hot glue gun type of device.

Another known repair procedure involves replacing the diseased or damaged nucleus pulposus with a prosthetic disc nucleus (PDN) while retaining the annulus fibrosis of an intervertebral disc. The PDN implant consists of a hydrogel core constrained in a woven polyethylene jacket (Raymedical, Inc., Bloomington, Minn.). The pellet-shaped hydrogel core is compressed and dehydrated to minimize its size prior to implant. After implantation, the hydrogel immediately begins to absorb fluid and expand. The tightly woven ultrahigh molecular weight polyethylene (UJMWPE) allows fluid to pass through to the hydrogel. This flexible but inelastic jacket permits the hydrogel core to deform and reform in response to changes in compressive forces yet constrains horizontal and vertical expansion upon hydration. The PDN implant's downside is that it takes approximately 4-5 days for the hydrogel to fully expand and reach its final dimension. Furthermore, although the PDN implant's hydrogel core is compressed and dehydrated into a small pre-implant package, a percutaneous placement has not been realized at the time of this writing because of the physical size of the PDN implants.

There are a variety of disadvantages to such techniques. Open surgical techniques generally require the use of general anesthesia, have a relatively long recovery time, and require the use of operating and recovery rooms. The procedure involves significant pain, a long recovery time, and the use of an expensive surgical facility. Thus, there exists a need for an improved surgical device that would allow a minimally invasive percutaneous method of repairing intervertebral discs in humans or other mammals.

### SUMMARY OF THE INVENTION

According to an aspect of the invention, a device for repair of mammalian intervertebral discs and/or articular joints is disclosed. The device is used to deliver or inject thermoplastic elastomer (TPE) material in a flowing liquid state to an intervertebral disc space or an articular join space. The device is generally used in conjunction with a cannula to percutaneously access the intervertebral disc space or an articular joint space for repair. The device comprises a catheter portion that is inserted through the cannula to deliver the TPE to the repair site.

The catheter comprises a distal end, a proximal end and a lumen extending longitudinally therethrough for delivery of a balloon inflating material. An expandable balloon having an opening may be detachably attached to the catheter near the distal end and defined within the balloon is a chamber which is in fluid communication with the lumen. The proximal end of the catheter is connected to an injector for holding a reservoir supply of a thermoplastic elastomer (TPE). The injector is configured and adapted with a heating element to maintain the TPE at an elevated temperature keeping the TPE in a flowing liquid state. The injector injects the liquid TPE through the catheter to an intervertebral disc space or an articular joint space. In an embodiment of the invention where the surgical device includes an expandable balloon, the TPE is used to inflate the balloon. In an embodiment of the invention where the surgical device does not include an expandable balloon, the TPE is directly injected into the surgical repair site.

The TPE is of a composition that is at a resilient and elastic solid in the temperature range of normal mammalian body temperature (i.e. human body temperature), but melts to a flowing liquid state at higher temperatures. The melting point temperature of the TPE will depend on the particular TPE composition and for the purpose of the invention, the TPE material should be of such composition whose melting point is substantially higher than the normal body temperature of the patient so that there is no chance of the TPE material ever re-melting after the completion of the surgical procedure of the invention.

The injector for the TPE material may be provided with a heated reservoir that maintains the TPE at the desired elevated temperature keeping the TPE in a liquid state. Alternatively, the injector may be configured more like a typical hot glue gun in which a solid TPE is melted by a heater just before being injected.

When repairing a diseased or damaged intervertebral disc using the device of the invention, the nucleus pulposus of the diseased or damaged intervertebral disc is first removed using any one of the known surgical techniques but preferably using a minimally invasive percutaneous procedure such as the DISC Nucleoplasty™ developed by ArthroCare Corporation. Preferably, the patient's spine should be put in traction in a horizontal position so that the intervertebral space is not under compression. A cannula is then used to gain access to the intervertebral space. Depending on the degenerated condition of the vertebral disc or the particular patient, an appropriate portion of the nucleus pulposus is removed through the cannula using a procedure such as the DISC Nucleoplasty™ mentioned above. The DISC Nucleoplasty™ technique allows removal of a precise amount of the nucleus pulposus material through a 17 gauge cannula, thus, minimizing the damage to the annulus of the disc.

The catheter of the TPE delivery device is inserted through the cannula and the heated fluid TPE material is injected into the intervertebral space substantially filling the void left behind by the removal of the nucleus pulposus. When the TPE cools to the human body temperature, it will be in its elastic solid sate, replacing the role of the original nucleus pulposus. The normal human body temperature is 37° C. and the TPE would harden into elastic solid at a temperature range between about 35° C. and 42° C.

When repairing a diseased or damaged intervertebral disc, the initial hot temperature of the fluid TPE material injected into the intervertebral space may provide the added therapeutic effect of reducing the pain in the area that was caused by the diseased or damaged intervertebral disc. This therapeutic effect of heat is a well known phenomenon.

According to an embodiment of the invention, the catheter may be provided with a detachable balloon at the distal end of the catheter for filling the intervertebral space with the TPE. The balloon may be formed of an expandable, flexible membrane. The interior of the balloon is in communication with the lumen of the catheter and the proximal end of the catheter is communicably attachable to the reservoir holding a supply of the TPE material.

According to another embodiment of the invention, the surgical device does not include the expandable balloon and the TPE material is directly injected into the intervertebral space via the catheter. The cannula will occlude the access through the annulus of the intervertebral disc while the TPE is still in liquid form. The catheter and the cannula may then be withdrawn after the TPE cools down to the patient's body temperature and the TPE solidifies into an elastic solid providing a replacement for the removed nucleus pulposus. The TPE material is preferably formulated to be radio-opaque so that the injection of the TPE material into the repair site may be monitored fluoroscopically.

According to another aspect of the invention, a surgical method of repairing intervertebral discs is also disclosed. First a cannula is used to percutaneously gain access to the intervertebral disc space to be repaired. The damaged or diseased nucleus pulposus may be removed through the cannula at this point using a procedure such as DISC Nucleoplasty™ or other percutaneous disc decompression procedures. A distal end of a catheter, having a detachable balloon attached thereto, is inserted through the cannula and into the nucleus space in the intervertebral disc left behind by the removed nucleus pulposus. Next, the balloon is inflated to a desired size by injecting the balloon with TPE material delivered through the catheter. Once the balloon is inflated to a desired size, the catheter is withdrawn. The liquid TPE is allowed to solidify before the cannula is withdrawn and the patient is taken off traction. The inflated balloon, left inside the intervertebral disc, will function as a replacement for the removed nucleus pulposus once the TPE cools to the patient's body temperature and solidifies to an elastic solid. The balloon is inflated to a size that would increase the thickness of the disc to a desired size that is sufficient to increase the intervertebral space back to a normal height.

The TPE material polymerizes to a resilient solid at the normal human body temperature but can be changed to a flowing liquid state at an elevated temperature above the normal human body temperature. Thus, the supply of the TPE material is maintained at the elevated temperature in its reservoir, and the inflating material can be pumped through the catheter's lumen and inflate the balloon during the surgical procedure. According to an aspect of the invention, the TPE material is also preferably radiopaque making the material visible on fluoroscope and allowing the surgical procedure to be performed under a fluoroscopic guidance for more precise control over the placement and the amount of the TPE material injected into the repair site.

According to another embodiment of the invention a method for percutaneous repair of an articular joint is disclosed. Access to the articular joint space is percutaeneously provided using a cannula. The removal of the degenerative cartilage may be conducted through the same cannula. A distal end of a catheter is then inserted through the cannula and into the articular joint. The catheter comprises a distal end and a proximal end and a lumen extending longitudinally therethrough for delivery of a balloon inflating material. An expandable balloon having an opening and a chamber defined therein is detachably attached to the catheter near the distal end. The internal chamber of the balloon is in fluid communication with the lumen. The opening of the balloon may be provided with a one-way valve that seals the chamber when the catheter is detached from the balloon. Once the distal end of the catheter with the balloon attached reaches the articular joint space, the TPE balloon inflating material is injected into the balloon until the balloon inflates to a desired size to function as a replacement for the cartilage. Once the procedure is complete, the catheter and the cannula are withdrawn from the patient, leaving the inflated balloon inside the articular joint. This technique may be applicable in hip, knee and shoulder joints.

The TPE delivery device of the present invention allows precise control over the amount of the TPE material injected to the surgical repair site. The delivery device is considerably smaller than such prior art devices as the hot glue gun described in the Ross patent, making it easier to handle in the operating room. Furthermore, the surgical repair procedure of the present invention is minimally invasive percutaneous procedure that does not require general anesthesia. Thus, this procedure may be performed as an outpatient procedure and as such can be provided at a considerably lower cost than any of the currently existing procedures.

### BRIEF DESCRIPTION OF THE DRAWINGS

FIG. 1A is a cross-sectional illustration of a surgical device according to an embodiment of the invention.

FIG. 1B is a cross-sectional illustration of an injector according to an embodiment of the invention which is part of the surgical device of FIG. 1A.

FIG. 2 is a cross-sectional view through the line A-A of the surgical device of FIG. 1.

FIG. 3A is an illustration of the expandable balloon that may be used with the surgical device of the invention in an uninflated folded configuration.

FIG. 3B is a top view of the expandable balloon of FIG. 3A in an inflated state.

FIG. 3C is a cross-sectional view through the line B-B of the expandable balloon of FIG. 3B being inflated with TPE 108 in a liquid state.

FIG. 3D is the cross-sectional view of the expandable balloon shown in FIG. 3C in an inflated state with the catheter detached.

FIG. 3E is the cross-sectional view of the expandable balloon shown in FIG. 3D after the TPE has cooled to an elastic solid state.

FIG. 4A is a perspective illustration of an intervertebral disc and its neighboring vertebral bodies.

FIG. 4B is a sagittal plane cross-sectional view of the vertebral structure of FIG. 4A.

FIG. 4C is a sagittal plane cross-sectional view of the vertebral structure of FIG. 4B after the intervertebral disc has degenerated.

FIGS. 5A-5C are sectional top views of an intervertebral disc illustrating the different stages of the disc repair surgical procedure using the device of the invention.

FIG. 6A is the sagittal plane cross-sectional view of FIG. 4B after the intervertebral disc has been repaired using an expandable balloon according to an aspect of the invention.

FIG. 6B is the sagittal plane cross-sectional view of FIG. 4B after the intervertebral disc has been repaired without the use of the expandable balloon according to another aspect of the invention.

FIG. 7A is an illustration of a normal hip joint.

FIG. 7B is an illustration of an arthritic hip joint.

FIG. 7C is an illustration of the hip joint of FIG. 7B whose damaged cartilage have been repaired according to an embodiment of the invention.

### DETAILED DESCRIPTION

The devices and methods according to the invention described herein are adapted for percutaneous surgical operation using cannulas and catheters. Referring to FIG. 1A, a surgical device 100 for percutaneous repair of intervertebral discs and articular joints according to an embodiment of the invention is disclosed. The surgical device 100 includes a cannula 120 for percutaneously accessing the surgical repair site, such as an intervertebral disc or an articular joint. A catheter 110 is disposed within the cannula 120. The catheter 110 comprises an elongated shaft having a proximal end 114 and a distal end 115 for delivery of a polymer-based balloon inflating material. A lumen 112 extends longitudinally through the catheter 110 for delivery of the polymer-based balloon inflating material. In a preferred embodiment of the invention, this balloon inflating material is a thermoplastic elastomer (TPE). An expandable balloon 150 may be detachably attached to the catheter 110 at its distal end 115. The balloon 150 is expandable and its interior chamber 152 is in fluid communication with the lumen 112.

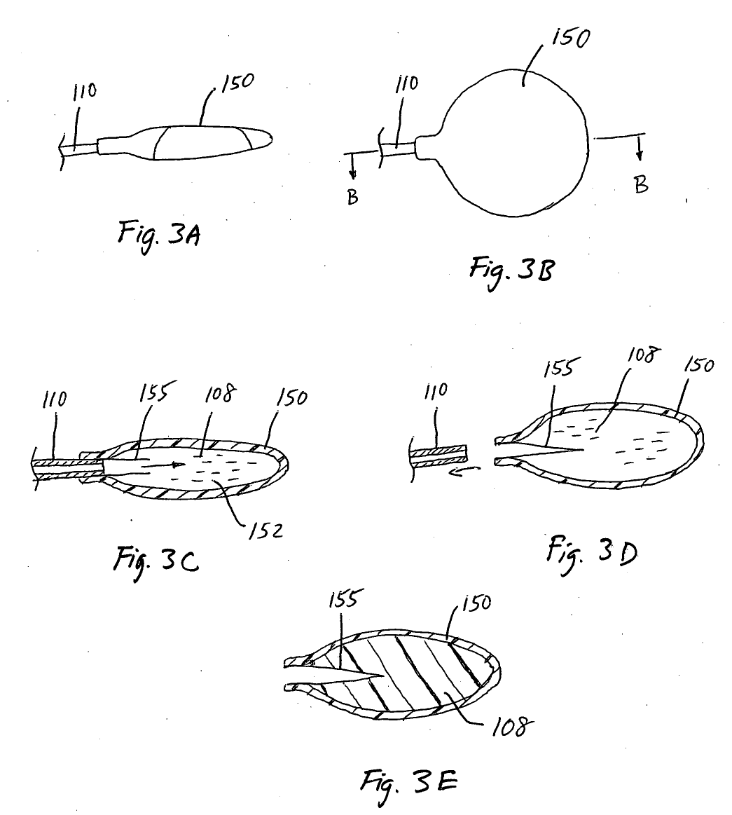

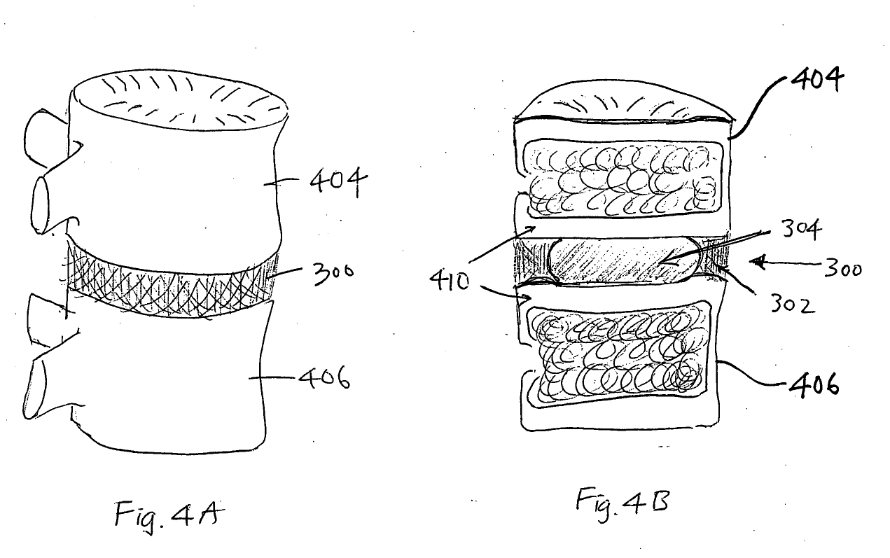

The catheter 110 may be of any suitable diameter depending on the needs of the particular application. For example, when repairing a human adult intervertebral disc, a catheter of suitable diameter to fit through an 11 or 13 gauge cannula would be appropriate. The balloon 150 may be made of a suitable material capable of withstanding the elevated temperature of the TPE during delivery and the extrinsic compression force exerted by the vertebral bodies. Because of this application requirement, the balloon 150 is preferably made from a semi-compliant material. This means that the balloon is made of a material such that before being inflated, the balloon 150 is not at its full dimension. But when inflated, the balloon 150 expands to take on a shape that is larger than the uninflated state and forms a predetermined shape. The predetermined shape preferably conforms to the particular surgical repair site into which the balloon is being implanted. For example, in the case of repairing an intervertebral disc, the balloon is generally placed into a void left behind after removal of the nucleus pulposus of the intervertebral disc and the balloon will have a predetermined inflated shape that substantially matches the void. The balloon would inflate to a flat disc shape with rounded edges. This exemplary shape of the inflated balloon 150 is illustrated in FIG. 3B. In the case of repairing an articular joint, the balloon is placed into the articular joint space to replace the diseased cartilage that has been removed from the articulating bone surface. A variety of synthetic polymers may be employed to provide a suitable balloon.

FIGS. 3A-3E provide more detailed illustrations of the balloon 150. A mitered valve 155 may be provided near the opening of the balloon that will allow the balloon inflating material 108 to flow in one direction only (i.e., into the balloon's internal chamber 152) and seals itself once the balloon 150 is inflated to a desired size and the catheter 110 is removed. A mitered valve is generally a bicuspid valve or a valve with two cusps or leaves. Normally, the leaves are separated and open at one end (i.e., the end near the mouth of the balloon) and lie together and remain closed at the opposite end (i.e., the end towards the inner space of the balloon). The valve 155 in closed state is illustrated in FIGS. 3D and 3E. When the catheter 110 is inserted into the mouth of the balloon 150 and the balloon inflating material 108 (i.e., the TPE material) is injected through the catheter 110, as illustrated in FIG. 3C, the pressure of the flowing material pushes the cusps or leaves of the valve 155 apart, thus opening the valve. Once the balloon 150 is inflated to a desired state and the flow of the inflating material is stopped, the pressure equalizes on both sides of the valve 155, the leaves close, preventing reversal of flow. As shown in FIG. 3D, the catheter 110 may then be removed, leaving the balloon 150 in a sealed state. The sealing of the mitered valve 155 maintains the balloon 150 at the inflated state. Such mitered valves have been employed in intravascular detachable balloons, such as those manufactured by Ingenor of France and Boston Scientific of United States. The catheter 110 and the mitered valve 155 opening of the balloon is assumed to be similar to those found on intravascular detachable balloons.

The proximal end 114 of the catheter 110 is preferably adapted and configured to be connected to an injector 200 that may house a supply of polymer-based inflating material 108. In an exemplary structure illustrated in FIG. 1, the proximal end 114 of the catheter 110 and the injector 200 are provided with couplers 103 and 203, respectively, for communicatingly coupling to one another. The couplers 103 and 203 may be a standard metal Luer-Lok® type coupling structures commonly found in the medical industry for angiographic devices and syringe/needle connections. When the catheter 110 is connected to the injector 200, the lumen 112 of the catheter 110 is in fluid communication with the injector 200 so that the inflating material 108 can be delivered to the balloon 150 via the lumen 112.

Referring to FIGS. 1B-1D, an embodiment of the surgical device 100 with a detailed illustration of injector 200 according to an embodiment of the invention is disclosed. The injector 200 allows the surgeon to accurately control the amount of the inflating material 108 that is delivered to the balloon 150 or directly into the intervertebral disc space, as described below according to another aspect of the invention. The injector 200 comprises a barrel 205 defined therewithin a chamber 215 for holding the inflating material 108. One end of the barrel is the coupler 203 with a channel 202 therethrough providing a passageway for the inflating material 108 from the chamber 215 to the catheter 110. A heater element 220 is incorporated into the barrel 205 and provides the heat necessary to maintain the inflating material 108 at a predetermined elevated temperature. The predetermined elevated temperature is sufficiently high to maintain the inflating material 108, which is preferably a thermoplastic elastomer, at a liquid state. The heater element 220 may be an electrical heating element and the barrel 215 may be provided with an electrical connector set 10. A similar electrical connector configuration 11 is also shown for the first heater element 50 of the catheter 110. The heater element 220 may be embedded within the walls of the barrel 205 as illustrated. Such barrel 205 can be made from injection molded plastic. Alternatively, the heater element 220 may be wrapped around the outside of the barrel 205.

A plunger 210 that is receivable within the barrel 205 is provided for providing the pressure necessary to deliver the inflating material 108 through the catheter 110 to the surgical repair site. The plunger 210 has a plunger head 211 at its distal end and a threaded shaft portion 212. The plunger 210 may also be provided with a winged handle 215 for ease of turning the plunger 210 to drive the plunger 210 and inject the inflating material 108. The threaded shaft portion 212 functions like a ball nut screw. The inside wall of the barrel 205 defining the chamber 215 has a threaded portion 205B, for mating with the threaded portion 212 of the plunger 210, and a non-threaded portion 205A for sealingly engaging the plunger head 211. The diameter of the plunger head 211 and the barrel's non-threaded portion 205A are appropriately sized to function like a syringe. To inject an amount of the liquid inflating material 108 through the catheter 110 and to the intervertebral disc or articular joint repair site, the plunger 210 is threaded into the barrel 205. The barrel 205 may also be provided with one or more handles 207 for ease of holding and manipulating the injector 200.

The inflating material 108 is preferably a TPE material that is in an elastic solid state in the temperature range of normal mammalian body temperature, such as a human patient's body temperature. But at temperatures above its melting point temperature, the TPE is in a flowable liquid state. The formulation of the TPE for this application is selected so that its melting point is higher than the range of normal mammalian body temperature. Examples of such material include medical grade TPE, thermoplastic polyethylenes, and thermoplastic polyurethanes. In its solid state, the TPE material should retain sufficient resiliency in order to provide adequate cushioning of the vertebrae or the articular joint.

In one embodiment, TPEs based on segmented polyurethanes (TPU) may be used. TPUs are polyurethane elastomers that are fully thermoplastic. It is a linear segmented block copolymer composed of hard and soft segments. TPUs are generally made from long chain polyols (poly-alcohols) with an average molecular weight of 600 to 4000, chain extenders with a molecular weight of 61 to 400, and polyisocyanates. The hard segment can be either aromatic or aliphatic in nature. The soft segment can be either polyether or polyester type. The choice affects the relative suitability for a given application. The polyurethane soft segments control low temperature properties, resistance to solvents, and the weather resistant properties of the TPUs. There are two types of flexible segments that are important: the hydroxyl terminated polyesters and the hydroxyl terminated polyethers.

For use in wet environments, for example, a polyether-based TPU is preferred. When oil and hydrocarbon resistance are primary factors, a polyester-based TPU is the material of choice. Another polyester type, polycaprolactone, also provides oil and hydrocarbon resistance with improved hydrolytic stability. A wide variety of property combinations can be achieved by varying the molecular weight of the hard and soft segments, their ratio and chemical composition. TPU excels in offering an effective and wide combination of physical properties and attributes over a range of hardness. TPUs offer high elasticity, high resiliency, good compression set, and flexibility without plasticizers, impact resistance (toughness), tear resistance and hydrolytic resistance. All of these properties make them ideal components for artificial disc replacement as used in this invention.

There have been many recent applications of TPUs in biological environments. TPUs were first integrated into biological environments in dental materials. Earlier studies have shown that TPUs are extremely tough, hydrolytically stable, non-toxic, non-carcinogenic, and very inert in a biological environment. It is also known that in a simulated body environment (37° C. n-saline), TPU material may soften. The softening of a given TPU material is a function of its composition, structure and resultant morphology. The softening of the TPU material is reversible and depends on the ratio of crystalline to amorphous segments and the extent of microphase separation. These parameters can be selected during the polymer synthesis and processing in order to control the degree of softening. By controlling these parameters, one can control the range of compressive and torsional properties of a given TPU composition enabling designing TPU compositions that have mechanical properties similar to the natural intervertebral disc.

The TPE material 108 is preferably maintained at a predetermined elevated temperature, the set temperature, that is higher than the melting point of the TPE, in the injector 200, so that the material is always in a flowable state ready to be injected. Thus, the injector 200 may be provided with a heating means to maintain the set temperature. The heating means may be any suitable heating device such as electrical heating element, along with any necessary temperature control circuits, that can maintain the temperature of the TPE material 108 at the set temperature.

Preferably, the set temperature of the TPE material 108 in the injector 200 is sufficiently high to compensate for any heat loss encountered by the TPE material 108 during its travel from the injector 200 to the balloon 150 to prevent the TPE material 108 from solidifying before reaching the balloon 150. Additionally, the set temperature of the TPE material 108 may be maintained sufficiently high so that by the time the TPE material 108 reaches the balloon 150, it is still hot enough to provide the pain reducing therapeutic effect to the patient. For example, the TPE material 108 may be a thermoplastic polymer composition that is in a liquid state at 100° C. because this temperature is optimal for providing the therapeutic pain reducing effect when injected in to the intervertebral disc.

In another embodiment, the catheter 110 may be provided with a heating element 50 along a substantial portion of its length to maintain the elevated temperature of the TPE material 108. In the example illustrated in FIG. 1A, the heating element 50 is an electrical heating wire wrapped around the catheter 110.

According to another aspect of the invention, the TPE material 108 may be radio-opaque so that it may be visible under fluoroscopic imaging. This allows the surgeon to monitor the surgical procedure fluoroscopically and visually monitor the delivery of the TPE material 108 to the repair site and also monitor the inflation of the balloon 150.

Because fluoroscopy generally utilizes X-rays generated by tungsten target, the radio-opacificity of the TPE material 108 may be achieved by doping the TPE material 108 with materials that have absorption coefficients similar to the range of tungsten. Tantalum is well known in the medical imaging industry for this purpose. The inventor was able to achieve successful results by adding tantalum to the TPE material used as the TPE material 108.

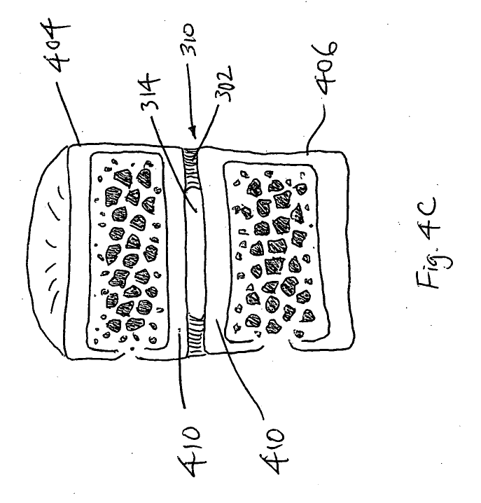

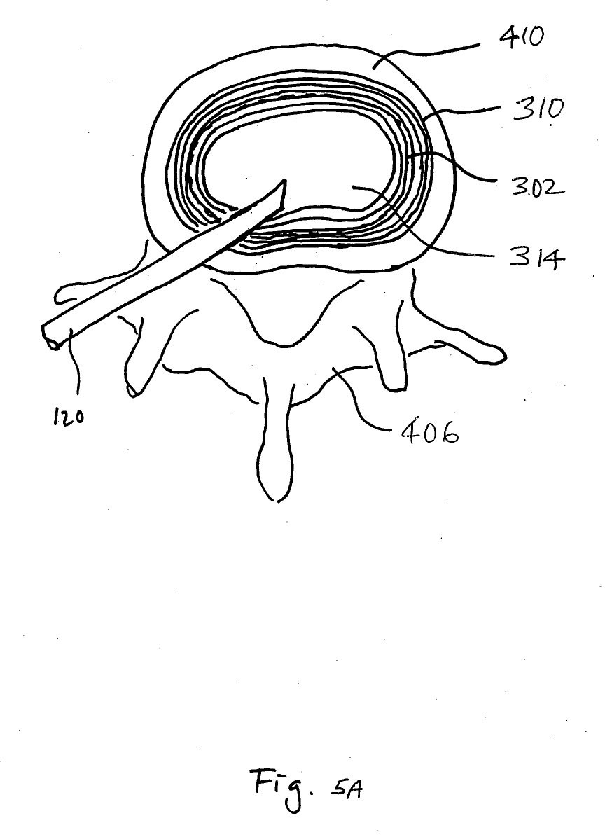

FIGS. 4A and 4B illustrate a portion of a healthy spinal column, specifically two vertebrae 404 and 406 with associated intervertebral disc 300. FIG. 4B illustrates a sectional view of the vertebrae 404, 406 sectioned in the sagittal plane. The intervertebral disc 300 located between the endplates 410 of the vertebrae 404, 406 comprises nucleus pulposus 304 and annulus fibrosus 302. FIG. 4C illustrates the vertebrae 404, 406 sectioned in the sagittal plane, after the intervertebral disc 300 has deteriorated into an unhealthy state. The deteriorated intervertebral disc 310 is substantially thinner than the healthy disc 300.

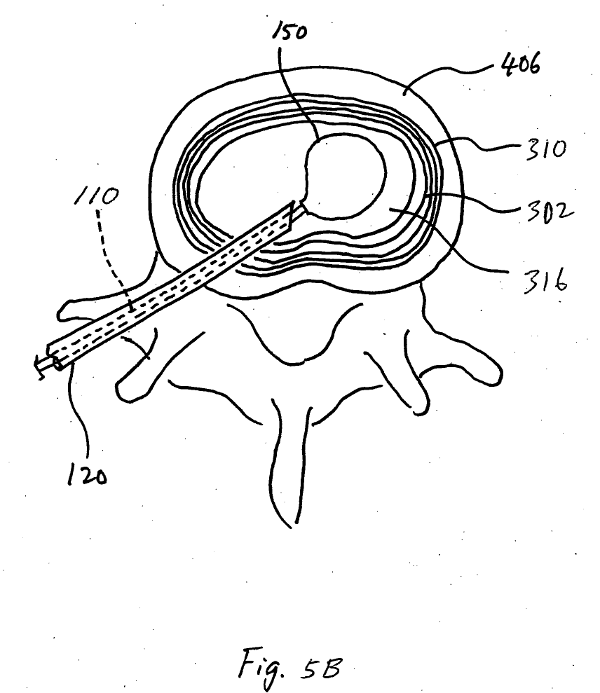

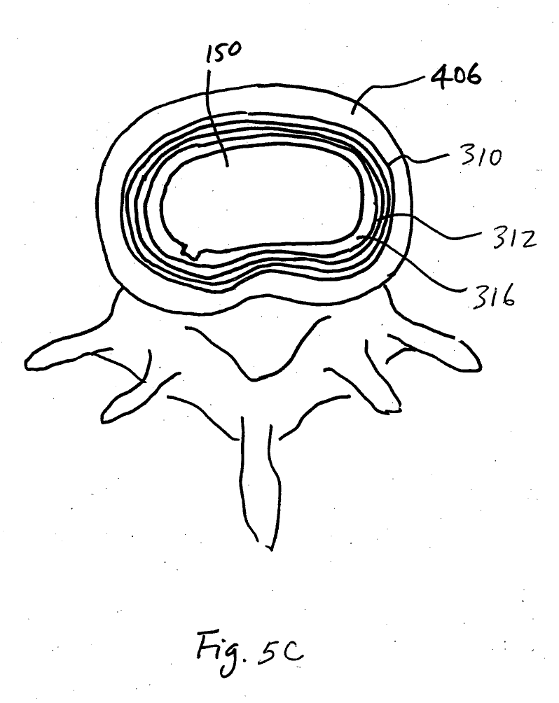

Referring to FIGS. 5A-5C, a surgical method of repairing an intervertebral disc 300 using the surgical device 100 according to an aspect of the invention will now be described. In this exemplary method, a method of repairing a damaged or deteriorated intervertebral disc 310 in a human is described. The same principles may be used for repair of intervertebral discs in other mammals. The problem in the deteriorated intervertebral disc 310 may typically be degenerated annulus fibrosus 312 and the nucleus pulposus 314 resulting in the whole disc structure being too narrow. The problem also may be a herniation of the nucleus pulposus 314.

In a process for repairing the diseased intervertebral disc 310 according to the present invention, the nucleus pulposus 314 of the deteriorated intervertebral disc 310 is removed, and a TPE material is percutaneously placed into the void left behind by the removal of the nucleus pulposus 314. In one embodiment of the invention, the expandable balloon 150 is percutaneously placed into the void using the surgical device 100 and inflated by injecting the TPE material into the balloon 150. In another embodiment of the invention, the TPE material is directly injected into the void without the use of the balloon 150.

Referring to FIGS. 5A and 5B, which are transverse plane sectional views of the diseased intervertebral disc 310, a cannula 120 may be used to percutaneously access and remove the nucleus pulposus 314. The removal of the nucleus pulposus 314 may be carried out by first dissecting the damaged nucleus pulposus 314, either with a mechanical discectomy device, electrocautary device, a laser device or a plasma tissue removal device and then aspirating the dissected tissue debris through the cannula 120. The removal of the diseased or damaged material provides a void 316 within the disc. It will be appreciated that in some instances a void may be present in the disc without removal of any damaged disc material.

In an embodiment where the balloon 150 is used, the balloon 150 attached to the distal end of a catheter 110 is inserted through the cannula 120 into the void 316 left behind by removal of the nucleus pulposus 314. When the balloon 150 is being inserted through the cannula 120, it may be folded as illustrated in FIG. 3A. The balloon 150 is inflated by injecting the TPE material in a liquid state through the catheter 110. As discussed above in reference to the injector 200, the TPE material is at an elevated temperature above its melting point and the elevated temperature may have a therapeutic pain reducing effect. The TPE material is formulated to be radio-opaque so that the surgical procedure may preferably be conducted while being fluoroscopically monitored. Because the TPE material can be visually monitored using a fluoroscope, the surgeon can inflate the balloon 150 to a desired state that will optimally fill the void 316.

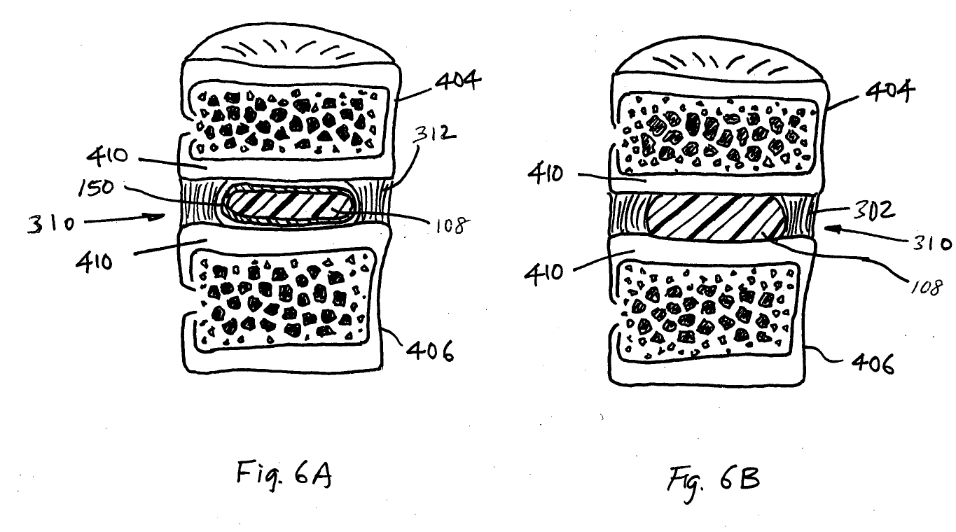

The balloon 150 may be provided in a variety of sizes and shapes to accommodate the various sizes and shapes of the void 316 in various patients. And for a particular case, the balloon 150 is preferably selected to have a size and shape that will restore the spacing between the two adjacent vertebrae to their normal state when the balloon 150 is fully inflated. FIG. 5C shows the inflated balloon 150 occupying the disc void 316. FIG. 6A is a sectional view in the sagittal plane of the repaired intervertegral disc 310 showing the inflated balloon 150 filled with the TPE material 108 after it has been cooled down to body temperature and have solidified. The thickness of the diseased intervertebral disc 310 has been restored to a healthy state.

This procedure may be further facilitated by placing the patient in traction for the procedure. The physician can monitor the balloon inflation process by monitoring the movement of the vertebral bones caused by the pressure of the inflating balloon 150. This allows the balloon 150 to fill the void 316 to a desired level and restore the spacing between the vertebrae 404 and 406 adjoining the intervertebral disc 300 to a normal healthy state. Generally, separation of the opposing endplates 410, the boney margins of the vertebra to which the intervertebral discs are attached, is desired. Therefore, the inflation of the balloon 150 is continued after the void 316 is filled, until the opposing endplates 410 of the vertebrae 404, 406 above and below the intervertebral disc 310 are separated by a desired amount. Thus, the surgeon can monitor the balloon filling process through fluoroscopic imaging, and can view the separation of the endplates. The surgeon can see when the cavity 206 is filled, and can see the movement of the vertebrae, and stop the flow of the TPE material 108 at the appropriate moment.

Once the desired separation is achieved, the flow of TPE material 108 is stopped and the catheter 110 is withdrawn from the balloon 150 and the catheter 110 and the cannula 120 are removed from the repair site. As discussed above in reference to FIG. 3D, when the catheter 110 is withdrawn from the balloon 150, the balloon's mitered valve 155 automatically seals the balloon retaining the TPE material 108 inside. The balloon 150, which is preferably made of an elastic material, and the catheter 110 are held together by friction between the mouth of the balloon and the catheter 110 and after the balloon is inflated inside the intervertebral disc void 316, the catheter can be removed simply by withdrawing it.

As illustrated in FIG. 6B, according to another embodiment of the invention, the repair of the deteriorated intervertebral disc 310 may be accomplished by injecting the TPE material 108 directly into the void 316 without using the balloon 150. In this embodiment, the catheter 110 is inserted through the cannula 120 and into the void 316. The liquid TPE material is then directly injected into the void 316. As before, this procedure is preferably monitored fluoroscopically so that injection of the radio-opaque TPE material 108 into the void 316 may be visually monitored. The flow of the TPE material 108 is continued after the void 316 is filled with the TPE material 108 until the opposing endplates of the vertebrae above and below the intervertebral disc 310 are separated by a desired amount. Once the desired separation is achieved, the flow of the TPE material 108 is stopped. The cannula 120 and the catheter 110 are preferably kept in place until the TPE material cools down sufficiently to solidify to prevent the liquid TPE from escaping the void 316. Once the TPE material cools to normal human body temperature, it does not flow and will remain within the annulus fibrosus 312. FIG. 6B illustrates the diseased intervertebral disc 310 that has been repaired according to the procedure of the present invention, whose thickness has been restored to a healthy state.

Whether the balloon 150 is used or not, as discussed previously, the TPE material 108 is kept at an elevated temperature in the injector 200 to keep the TPE material 108 in a liquid state for injection into the repair site. After the TPE material 108 is injected into the repair site directly or into the balloon 150 that has been placed in the repair site, the heat from the hot TPE material 108 will dissipate to the surrounding surfaces. This application of heat is salutary. The therapeutic effect of applying heat to intervertebral space to ameliorate pain associated with diseased or deteriorated disc is well known in the art.

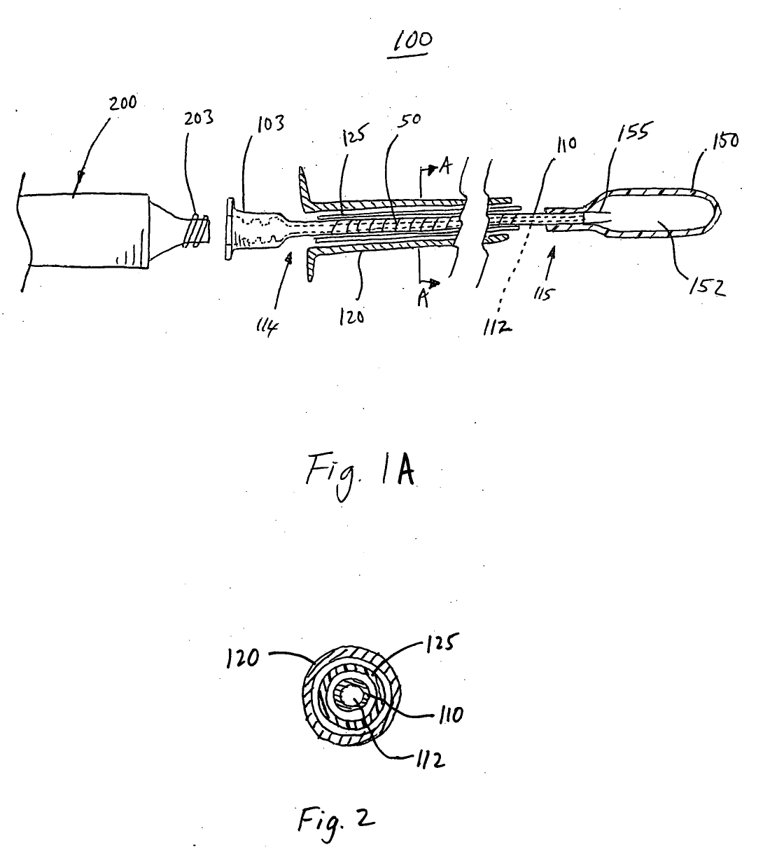

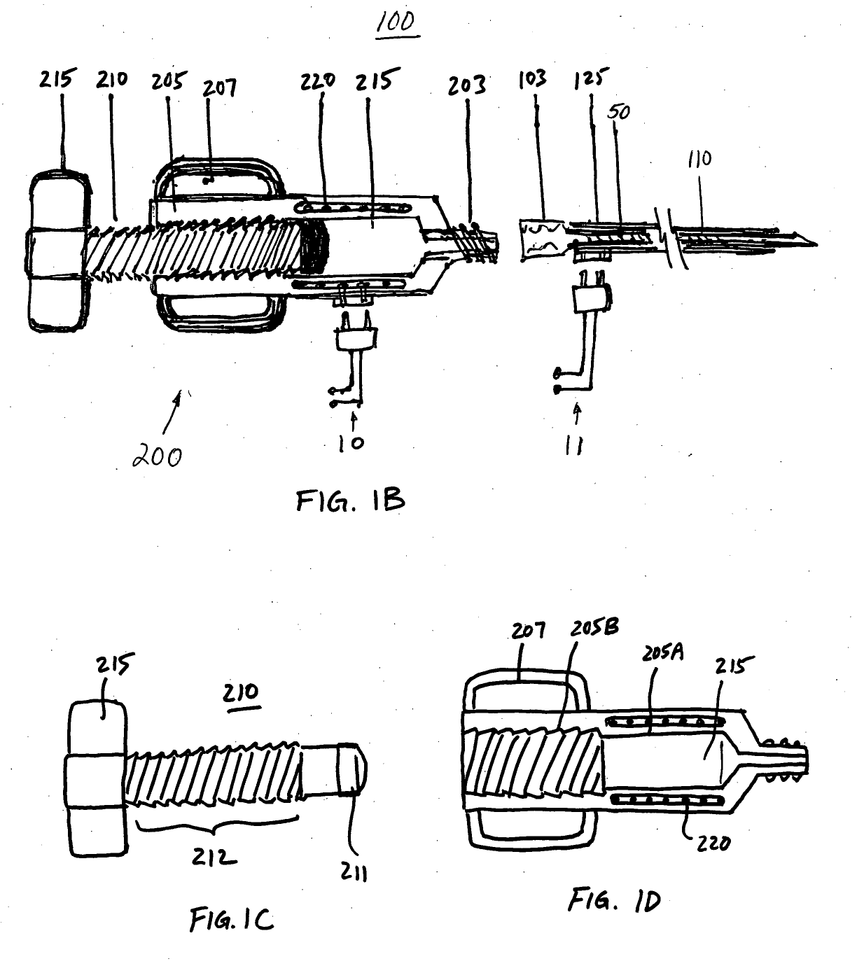

As shown in FIGS. 1 and 2, according to another embodiment of the invention, an optional heat insulating shield 125 may be used to insulate the cannula 120 from the hot inflating material (TPE) 108 flowing through the catheter 110. This prevents the cannula 120 from getting too hot for the surrounding tissues. The insulating shield 125 may be an elongated tubular structure made from a suitable heat insulating material. Its diameter is smaller than the inside diameter of the cannula 120 and larger than the outer diameter of the catheter 110 so that the insulating shield 125 can fit in between the catheter 110 and the cannula 120. In an embodiment where the insulating shield 125 is used, the diameters of the cannula 120, the insulating shield 125, and the catheter 110 are such that the catheter's fit within the cannula 120 and the insulating shield 125 is maintained at an optimal level and not too tight and not too loose. The catheter 110, the insulating shield 125, and the cannula 120 should fit as closely as possible so that the catheter 110 can be withdrawn from the assembly while the cannula 120 remains in place. The outer diameter of the assembly will be the smaller size possible that will still accommodate the shield 125 and catheter 110 of a gauge large enough to allow sufficient passage of the heated liquid TPE through the catheter 110. The thickness of the insulating shield 125 would be adjusted according to the needs of a particular application. Polymers such as Kevlar or Mylar are good examples of such heat insulating material. Alternatively, the cannula 120 itself may be made from a non-metallic thermally insulating material.

Referring to FIG. 4, a detailed view of the injector 200 according to an embodiment of the invention is illustrated.

The described device and technique may also be applied to other joints. For example, deteriorated cartilage in articulating joints such as the knee, shoulder and hip may be replaced using this device and technique. In this second application of the invention, a balloon filled with the TPE material will be used to replace degenerated or diseased cartilage on articular bone surfaces that may have been destroyed by arthritis or trauma. For the articular joint application, the expandable balloon is shaped to conform to the contour of the particular joint being repaired. The particular shape of the balloon will, when filled with the polymer-based inflating material, take the form of the cartilage on the articular bone surface. As in the case of intervertebral discs, the invention allows percutaneous removal and replacement of the damaged cartilage in the articular joint.

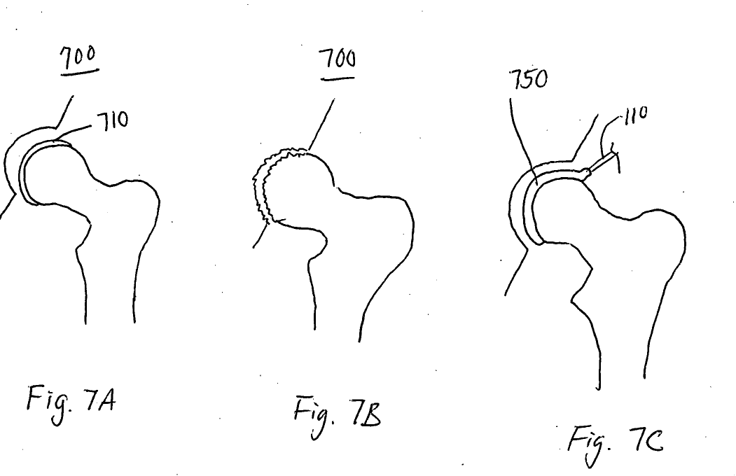

FIG. 7A is an illustration of a normal hip joint 700. FIG. 7B is an illustration of a hip joint in which the cartilages 710 on the articular bone surfaces have been destroyed by arthritis. FIG. 7C is an illustration of a balloon 750, shaped to fit the contour of the hip joint's articular bone surfaces. The balloon 750 would be introduced to the joint space percutaneously and filled with molten TPE material to a desired level, similar to the procedure described above in reference to the intervertebral disc application.

Various advantages are evident from the foregoing description. The percutaneous nature of the procedure is advantageous for reasons, such as faster recovery time, the absence of a need for extensive surgical procedures, and the like, known in the art. As the procedure is conducted percutaneously and is fluoroscopically monitored, the process can be conducted in a radiology department or radiological facility on an outpatient basis, rather than in a surgical facility.

Although the invention has been described in terms of exemplary embodiments, it is not limited thereto. Rather, the appended claims should be construed broadly, to include other variants and embodiments of the invention, which may be made by those skilled in the art without departing from the scope and range of equivalents of the invention.

1. A surgical device for percutaneous repair of intervertebral discs or articular joints, the device comprising:
a catheter for inserting through a cannula, wherein the cannula percutaneously providing access to an intervertebral disc or articular joint, the catheter comprising:
a distal end and a proximal end;
a lumen extending longitudinally therethrough for delivery of a thermoplastic elastomer material; and
an injector, communicably coupled to the proximal end of the catheter, for delivering a supply of the thermoplastic elastomer material at a predetermined elevated temperature.

2. The device of claim 1, further comprising a heating element incorporated into the catheter for providing heat to a substantial portion of the lumen, wherein the thermoplastic elastomer material is kept in the predetermined elevated temperature.

3. The device of claim 2, wherein the heating element is wrapped around the catheter.

4. The device of claim 1, further comprising an expandable balloon having an opening detachably attached to the catheter near the distal end and an internal chamber which is in fluid communication with the lumen, wherein the opening of the balloon being provided with a one-way valve that seals the chamber when the catheter is detached from the balloon.

5. The device of claim 1, further comprising a heat insulating shield disposed between the cannula and the catheter.

6. The device of claim 1, wherein the thermoplastic elastomer is thermoplastic polyurethane.

7. The device of claim 6, wherein the thermoplastic elastomer is polyether thermoplastic polyurethane, polyester thermoplastic polyurethane, or a combination of both.

8. The device of claim 1, wherein the thermoplastic elastomer is at an elastic solid state at normal human body temperature and at a readily flowing liquid state at a predetermined temperature above the normal human body temperature.

9. The device of claim 1, wherein the thermoplastic elastomer is radio-opaque.

10. The device of claim 1, wherein the injector comprises:
a barrel for holding the thermoplastic elastomer material;
a second heater element incorporated into the barrel for heating and maintaining the temperature of the thermoplastic elastomer sufficiently high to keep the thermoplastic elastomer in liquid state; and
a plunger receivable within the barrel and having a shaft, the shaft having a threaded portion, wherein the barrel has threaded portion on its internal wall for mating with the threaded portion of the plunger's shaft.

11. The device of claim 10, wherein the second heater element is embedded within the barrel.

12. The device of claim 10, wherein the second heater element is wrapped around the barrel.

13. A method for percutaneous repair of an intervertebral disc of a patient, comprising the steps of:
(a) removing a nucleus pulposus of an intervertebral disc percutaneously, leaving behind a void inside the intervertebral disc;
(b) providing an access to the void percutaeneously using a cannula;
(c) inserting a distal end of a catheter through the cannula and into the void in the intervertebral disc, wherein the catheter comprises:
a distal end and a proximal end;
a lumen extending longitudinally therethrough for delivery of a thermoplastic elastomer material;
(d) injecting the thermoplastic elastomer material that is at a predetermined elevated temperature sufficiently high to maintain the thermoplastic elastomer material at a liquid state into the void using an injector, wherein the injector comprises:
a barrel for holding the thermoplastic elastomer material;
a second heater element incorporated into the barrel for heating and maintaining the temperature of the thermoplastic elastomer sufficiently high to keep the thermoplastic elastomer in liquid state; and
a plunger receivable within the barrel and having a shaft, the shaft having a threaded portion, wherein the barrel has threaded portion on its internal wall for mating with the threaded portion of the plunger's shaft; ;and
(e) withdrawing the catheter and the cannula from the patient, wherein when the thermoplastic elastomer cools to the patient's body temperature, the thermoplastic elastomer solidifies into its elastic solid phase.

14. The method of claim 13, wherein the thermoplastic elastomer material is radio-opaque and the method is monitored fluoroscopically.

15. A method for percutaneous repair of an intervertebral disc of a patient, comprising the steps of:
(a) removing a nucleus pulposus of an intervertebral disc percutaneously, leaving behind a void inside the intervertebral disc;
(b) providing an access to the void percutaeneously using a cannula;
(c) inserting a distal end of a catheter through the cannula and into the void in the intervertebral disc, wherein the catheter comprises:
a distal end and a proximal end;
a lumen extending longitudinally therethrough for delivery of a balloon inflating material;
an expandable balloon having an opening detachably attached to the distal end of the catheter, the balloon defining a chamber which is in fluid communication with the lumen;
the opening of the balloon being provided with a one-way valve that seals the chamber when the catheter is detached from the balloon;
(d) injecting the balloon inflating material that is at a predetermined elevated temperature sufficiently high to maintain the balloon inflating material at a liquid state into the balloon using an injector until the balloon inflates to a desired size, wherein the injector comprises:
a barrel for holding the thermoplastic elastomer material;
a second heater element incorporated into the barrel for heating and maintaining the temperature of the thermoplastic elastomer sufficiently high to keep the thermoplastic elastomer in liquid state; and
a plunger receivable within the barrel and having a shaft, the shaft having a threaded portion, wherein the barrel has threaded portion on its internal wall for mating with the threaded portion of the plunger's shaft; and
(e) withdrawing the catheter and the cannula from the patient leaving the inflated balloon inside the intervertebral disc, wherein when the balloon inflating material cools to the patient's body temperature, the balloon inflating material solidifies into its elastic solid phase.

16. The method of claim 15, wherein the balloon inflating material is radio-opaque and the method is monitored fluoroscopically.

17. A method for percutaneous repair of an articular joint of a patient, comprising the steps of:
(a) percutaneously removing degenerative cartilage from the articular joint;
(b) providing an access to the articular joint percutaeneously using a cannula;
(c) inserting a distal end of a catheter through the cannula and into the articular joint, wherein the catheter comprises:
a distal end and a proximal end;
a lumen extending longitudinally therethrough for delivery of a balloon inflating material;
an expandable balloon having an opening detachably attached to the catheter near the distal end and defining a chamber which is in fluid communication with the lumen;
the opening of the balloon being provided with a one-way valve that seals the chamber when the catheter is detached from the balloon;
(d) injecting the balloon inflating material that is at a predetermined elevated temperature sufficiently high to maintain the balloon inflating material at a liquid state into the balloon until the balloon inflates to a desired size; and
(e) withdrawing the catheter and the cannula from the patient, leaving the inflated balloon inside the articular joint.

18. The method of claim 17, wherein the balloon inflating material is radio-opaque and the method id monitored fluoroscopically.

19. The method of claim 17, wherein the balloon is shaped to conform to the articular joint's contour.
